(19) United States
(12) Patent Application Publication
(10) Pub. No.: US 2009/0281428 A1
(43) Pub. Date: Nov. 12, 2009

(54) Method and apparatus for examining a body with an ultrasound head

(75) Inventors: Sergej Kammerzell, Freilassing (DE); Markus Heller, Berlin (DE); Alexey Sharenkov, Berlin (DE); Christian Koenig, Berlin (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/386,003

(22) Filed: Apr. 9, 2009

(30) Foreign Application Priority Data
May 10, 2008 (DE) 10 2008 023 218

(51) Int. Cl. A61B 8/14

(52) U.S. Cl. 600/459

(57) ABSTRACT

In a method for extracorporeal examination of a body with an ultrasound head, in which a plurality of ultrasound emitters which are placed next to one another and emit ultrasonic radiation in a common central plane of the ultrasound head, and a plurality of ultrasound receivers are arranged in a row, in order to improve the reproducibility of the orientation of the ultrasound head, it is proposed that with the use of prominent points of the body, a geometrical structure which is defined by these prominent points be determined, and the ultrasound head be positioned relative to this geometrical structure in a defined orientation in which the ultrasound examination is performed on the body. An apparatus for performing this method is also described.

The present disclosure relates to the subject matter disclosed in German application number 10 2008 023 218.1 of May 10, 2008, which is incorporated herein by reference in its entirety and for all purposes.

### BACKGROUND OF THE INVENTION

The invention relates to a method for extracorporeal examination of a body with an ultrasound head, in which a plurality of ultrasound emitters which are placed next to one another and emit ultrasonic radiation in a common central plane of the ultrasound head, and a plurality of ultrasound receivers are arranged in a row.

The examination of body structures with ultrasound is a standard method enabling determination of the position and shape of a body structure, for example, the position of the front iliac crests and the pubic bone on the pelvis. The examination is normally carried out with an ultrasound head in which quite a large number of ultrasound emitters and ultrasound receivers which emit and receive ultrasonic radiation in a common central plane of the ultrasound head are arranged in a row. During the examination, a person guiding the ultrasound head can incline it in relation to the body so that the structure to be examined, for example, the iliac crest, is optimally represented on a display of the ultrasound examination device. However, inaccuracies in the localizing of the geometrical structures of the body occur owing to the fact that the ultrasound head may be differently orientated relative to the body, i.e., the reproducibility is limited.

An object of the invention is to so improve a generic method that the reproducibility of the examination results is increased.

### SUMMARY OF THE INVENTION

This object is accomplished, in accordance with the invention, in a method of the kind described at the outset, wherein with the use of prominent points of the body, a geometrical structure which is defined by these prominent points is determined, and the ultrasound head is positioned relative to this geometrical structure in a defined orientation in which the ultrasound examination is performed on the body.

Accordingly, during the examination the orientation of the ultrasound head is no longer freely selectable and determined solely by the quality of the representation on a display device, but rather this orientation is limited by the ultrasound head being able to assume only a certain orientation in relation to a geometrical structure of the body, however, slight deviations from this prescribed orientation of the ultrasound head may, of course, be tolerable.

This method results in the ultrasound head assuming a similar orientation relative to the body in all examinations, even though the position of the ultrasound head can be varied, i.e., the ultrasound head is displaced substantially parallel to itself on the body surface in order to examine the body structures.

The geometrical structure defined by the prominent points can be determined before the actual ultrasound examination, and the prominent points can be determined in an optional way, for example, using a navigated palpation instrument or an ultrasound head, which is then freely orientated in the conventional way relative to the body and at first does not assume the prescribed preferential orientation relative to the body structure. The results of this determination of the position of the prominent points of the body do not need to be very precise, such an approximate determination of the position of the prominent points being sufficient. Selected geometrical structures can then be determined with the position data of these prominent points. For example, these geometrical structures can be formed by a straight line joining the two prominent points to each other or by a plane spanned by three such prominent points.

The straight line joining the proximal joint head of a femur and a point indicating the position of the knee joint, for example, palpated through the position of the patella, can be given as example of a straight line. The determination of the frontal plane of the pelvis, determinable by palpating the upper iliac crests and the pubic bone, can serve as example of a geometrical structure in the form of a plane, always with the limitation that this position determination is only an approximate determination of the position of these geometrical structures, as the determination of the position of the prominent points is carried out with a relatively low degree of precision.

Nevertheless, geometrical structures are obtained in this way, for example, a straight line or a plane, which are characteristic of the body to be examined. The ultrasound head is now aligned in relation to these geometrical structures so that the central plane of the ultrasound head assumes a certain orientation relative to the geometrical structures, for example, extends perpendicularly to a straight line joining two prominent points or to a plane spanned by three prominent points. A defined orientation of the ultrasound head, which, at the most, differs from an ideal orientation by a few degrees owing to the imprecision of the prominent points is thereby ensured for the examination. At any rate, the margin for the orientation of the ultrasound head is in this way substantially limited in comparison with the margin allowed to a person carrying out the examination with conventional methods where no specifications exist for the orientation of the ultrasound head.

The invention also relates to an apparatus for examining a body, comprising an ultrasound head, in which a plurality of ultrasound emitters which are placed next to one another and emit ultrasonic radiation in a common central plane of the ultrasound head, and a plurality of ultrasound receivers are arranged in a row, a navigation system for determining the position of marker elements secured to the body, the ultrasound head and possibly further instruments, and a data processor which is fed the position data determined by the navigation system.

An object of the invention is to so design such an apparatus that improved reproducible ultrasound examination data are obtainable with it.

This object is accomplished, in accordance with the invention, in an apparatus of this kind in that the data processor is programmed so as to determine the orientation of the ultrasound head relative to the orientation of a geometrical structure of the body, which is defined by prominent points of the body, and the position data of which are stored in the data processor. The data processor thus calculates the orientation of the ultrasound head relative to the orientation of the geometrical structure, which has been determined by preliminary examinations, so that the user then has the possibility of orientating the ultrasound head relative to the geometrical structure as desired.

In particular, the data processor can be programmed so as to feed to a display device a signal which corresponds to a deviation in the orientation of the ultrasound head from the stored orientation of the geometrical structure. The user thus has the possibility of following on the display device the deviation of the orientation of the ultrasound head from the desired orientation, i.e., from a certain orientation relative to the orientation of the geometrical structure stored in the data processor.

The following description of preferred embodiments of the invention serves, in conjunction with the drawings, to provide a more detailed explanation.

### BRIEF DESCRIPTION OF THE DRAWINGS

FIG. 1 shows a schematic representation of a patient on an operating table with a navigation system and a navigated ultrasound head for performing ultrasound examinations;

FIG. 2 shows a schematic representation of a femur bone with a geometrical structure in the form of a straight line joining the head of the femur and a condyle, and an ultrasound head orientated perpendicularly to this straight line; and

FIG. 3 shows a schematic representation of a pelvic bone with a frontal plane spanned by the two upper iliac crests and the pubic bone, and an ultrasound head orientated perpendicularly to this frontal plane.

### DETAILED DESCRIPTION OF THE INVENTION

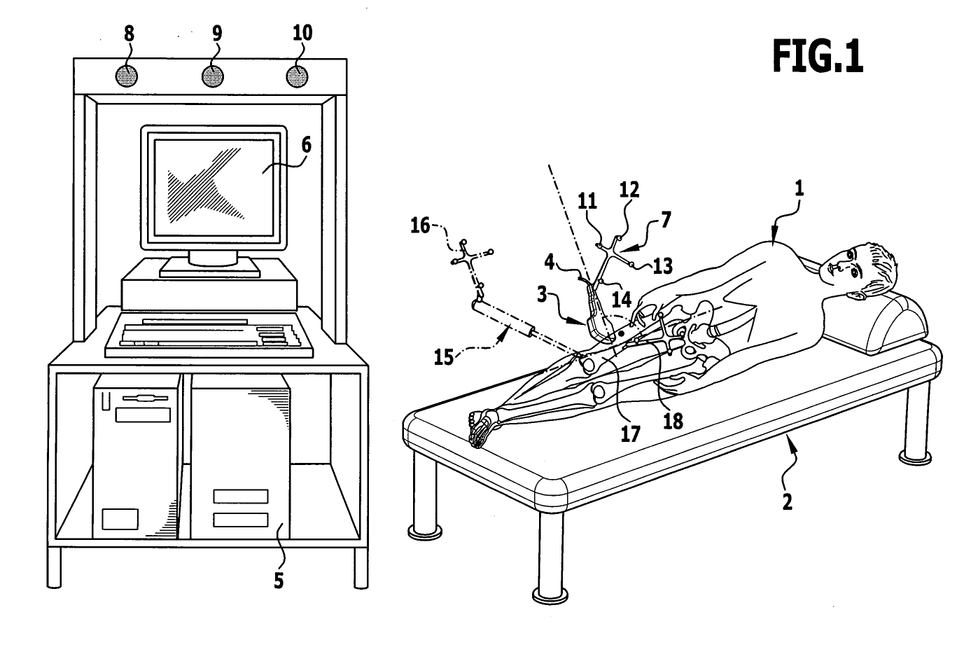

FIG. 1 shows a patient 1 lying on an operating table 2, on whom an ultrasound examination is to be performed. An ultrasound head 3 is used for this purpose. A plurality of ultrasound emitters which are placed next to one another and a plurality of ultrasound receivers are arranged in a row in the ultrasound head 3. These ultrasound emitters emit in a common central plane of the ultrasound head 3 ultrasonic waves which penetrate the body, are reflected there and are then received again by the ultrasound receivers on the ultrasound head 3. From the transit time of the ultrasonic radiation between emission and reception it is possible to determine the distance from a structure of the body at which the ultrasonic radiation is reflected. This applies to all ultrasound emitters and ultrasound receivers in the ultrasound head 3.

The data obtained in this way are fed through a line 4 to a data processor 5 comprising inter alia a display device 6 in the form of a monitor. The transit times of the ultrasonic radiation are represented on this display device 6 in such a way that an image of the structure reflecting the ultrasonic radiation is thereby produced.

Rigidly attached to the ultrasound head 3 is a marker element 7 which is part of a navigation system. This navigation system includes inter alia three emitters 8, 9, 10 spaced from one another, which emit a radiation reflected at reflection elements 11, 12, 13, 14 of the marker element 7 and pick it up again. Use of three spatially separate emitters 8, 9, 10 allows the distance of the reflection elements 11, 12, 13, 14 from the emitters to be determined and hence the position and the orientation of the marker element 7 relative to the emitters 8, 9, 10, i.e., in a fixed coordinate system. Since the marker element 7 is secured to the ultrasound head 3, a precise determination of the location of the ultrasound head 3 in the fixed coordinate system is also obtained, i.e., the respective position and the orientation of the ultrasound head 3.

The position data of the ultrasound head 3 determined by the navigation system can also be fed to the data processor 5.

FIG. 1 also shows a palpation instrument 15 which is also connected to a marker element 16 so that the navigation system is also able to determine the position of the palpation instrument 15.

It is also possible to attach such a marker element 18 to the femur 17 of the patient 1, for example, using a bone screw or a band around the patient's thigh, thereby eliminating the need for invasive surgery. In this way, the position of the femur in the fixed coordinate system can be determined.

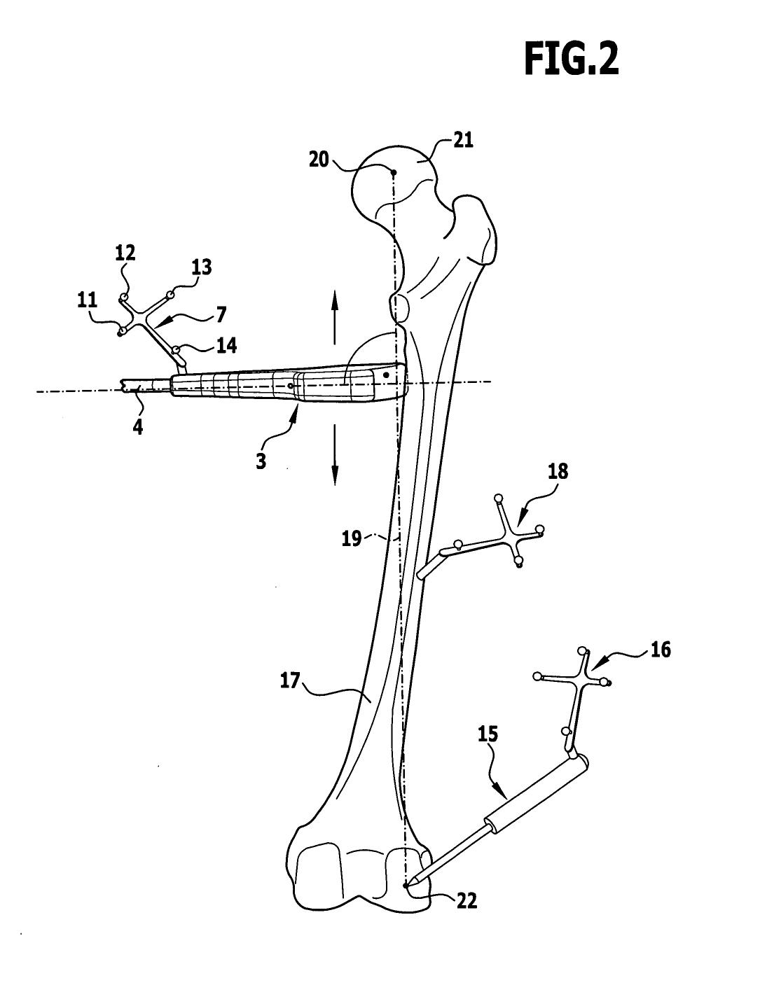

To examine the patient with an ultrasound head 3, the position of a selected geometrical structure in the body is first determined. The exemplary embodiment of FIG. 2 shows the patient's femur 17 in isolation and as selected geometrical structure a straight line 19 joining the midpoint 20 of the head 21 of the femur and a selected location 22 on a condyle in the proximity of the knee joint. The midpoint 20 and the selected location 22 thus form prominent points of the body, which can be determined in various ways, for example, by X-rays, CT scans, palpation—possibly through the skin—or by the movement of the femur 17 in the hip joint. For example, the midpoint 20 can be determined by the femur 17 being pivoted relative to the pelvic bone, during which the movement of the femur 17 can be determined by the marker element 18 fixed to it, and the path of movement of the marker element 18 then lies on the surface of a sphere whose midpoint is formed by the midpoint 20.

The selected location 22 in the area of the knee joint can, for example, be palpated through the skin using a palpation instrument 15. Alternatively, the position of the patella could be selected, it merely being essential that by determining two prominent points of the body, a geometrical structure of the body can be determined, in this case, a straight line 19, which joins these two prominent points and runs approximately along the femur 17.

The orientation of this straight line 19 relative to the femur 17 is stored in the memory of the data processor 5.

When examining the patient with the ultrasound head, the ultrasound head 3 is orientated in such a way that a certain orientation of the ultrasound head 3 relative to the stored geometrical structure is always assumed, for example, the ultrasound head 3 is always orientated in such a way that its central plane extends perpendicularly to the straight line 19, as shown in FIG. 2. The user can follow this orientation on the display device 6 which indicates in addition to the respectively captured ultrasonic image of the examined body structure the respective deviation of the orientation of the ultrasound head 3 from the orientation of the geometrical structure of the body, for example, the angle of the central plane of the ultrasound head 3 relative to the straight line 19. If this angle is at precisely 90°, the ultrasound head 3 is correctly orientated, otherwise an adjustment is necessary. During the examination, the ultrasound head 3 is then displaced parallel to itself in this orientation on the body surface until an optimum representation of the body structure to be examined is found. Since the orientation of the ultrasound head relative to the straight line 19 is maintained, a defined orientation of the ultrasound head 3 relative to the femur 17 is also obtained. Here it is not essential that the straight line 19 be formed exactly by prominent points whose position data are determined with a high degree of precision, as deviations of the position data of the prominent points result in only a slight change in the orientation of the straight line 19. Nevertheless, it is ensured in this way that the ultrasound head 3 is essentially always guided with the same orientation along the femur 17.

Moreover, the position data of the selected points could also be determined in a different way, for example, with the ultrasound head 3 itself. The position of the body's structures is first determined, i.e., for example, the femur head and the condyles, in any orientation of the ultrasound head 3. On the basis of this rough determination, without any prescribed orientation of the ultrasound head 3, prominent points can be reasonably accurately determined and, therefore, on the basis of this determination, the orientation of the straight line 19 can also be defined. Since the accuracy is not of any great importance in this case, these points can also be obtained when the ultrasound head 3 is at an incline in relation to the straight line 19.

During the final examination, however, care is taken to ensure that the ultrasound head maintains the prescribed orientation in relation to the thus predetermined and stored straight line 19.

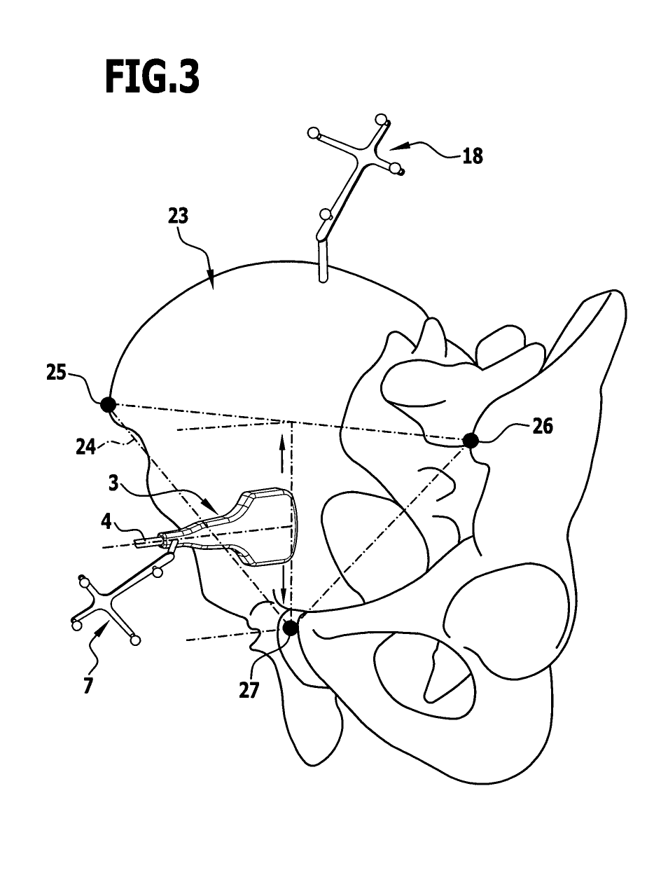

A similar procedure can be used when determining the position of the frontal plane of a pelvic bone 23 (FIG. 3). The frontal plane 24 of the pelvic bone 23 is usually determined by determining three prominent points of the pelvic bone 23, namely the two upper iliac crests 25, 26 and the pubic bone 27. These points can first be determined with a relatively low degree of precision using the ultrasound head 3, with the ultrasound head being orientated at random in relation to the front plane, it merely being essential to determine the approximate position of the prominent points. These are stored in the data processor 5 and, consequently, also the position data of the frontal plane 24.

During the final ultrasound examination, the ultrasound head 3 is then orientated such that its central plane extends perpendicularly to this predetermined frontal plane 24. With this orientation, the prominent points, i.e., the iliac crests 25, 26 and the pubic bone 27, can be determined with increased precision, thereby enabling an exact determination of the frontal plane 24.

In many cases, an orientation of the ultrasound head, which extends perpendicularly to a plane or a straight line, for example, perpendicularly to the frontal plane, is favorable. There may, however, also be other cases where another orientation is favorable, for example, an orientation of 75° relative to the frontal. plane. This depends on the anatomical features. It is merely essential that it be made possible for the operator to align the ultrasound head in a defined manner relative to a certain geometrical structure.

1. Method for extracorporeal examination of a body with an ultrasound head, in which a plurality of ultrasound emitters which are placed next to one another and emit ultrasonic radiation in a common central plane of the ultrasound head, and a plurality of ultrasound receivers are arranged in a row, wherein with the use of prominent points of the body, a geometrical structure which is defined by these prominent points is determined, and the ultrasound head is positioned relative to this geometrical structure in a defined orientation in which the ultrasound examination is performed on the body.

2. Method in accordance with claim 1, wherein the prominent points are determined with a navigated examination instrument.

3. Method in accordance with claim 2, wherein the prominent points are determined with a navigated palpation instrument.

4. Method in accordance with claim 2, wherein the prominent points are determined with a navigated ultrasound head.

5. Method in accordance with claim 1, wherein the geometrical structure is a straight line joining two prominent points of the body.

6. Method in accordance with claim 5, wherein the central plane of the ultrasound head is orientated perpendicularly to the straight line.

7. Method in accordance with claim 1, wherein the geometrical structure is a plane which is spanned by three prominent points of the body.

8. Method in accordance with claim 7, wherein the central plane of the ultrasound head is orientated perpendicularly to the plane.

9. Apparatus for examination of a body, comprising an ultrasound head, in which a plurality of ultrasound emitters which are placed next to one another and emit ultrasonic radiation in a common central plane of the ultrasound head, and a plurality of ultrasound receivers are arranged in a row, a navigation system for determining the position of marker elements secured to the body, the ultrasound head and possibly further instruments, and a data processor which is fed the position data determined by the navigation system, wherein the data processor is programmed so as to determine the orientation of the ultrasound head relative to the orientation of a geometrical structure of the body, which is defined by prominent points of the body and the position data of which are stored in the data processor.

10. Apparatus in accordance with claim 9, wherein the data processor is programmed so as to feed to a display device a signal which corresponds to a deviation in the orientation of the ultrasound head from the stored orientation of the geometrical structure.

11. Apparatus in accordance with claim 9, wherein the geometrical structure is a straight line joining two prominent points of the body.

12. Apparatus in accordance with claim 10, wherein the geometrical structure is a straight line joining two prominent points of the body.

13. Apparatus in accordance with claim 9, wherein the geometrical structure is a plane which is spanned by three prominent points of the body.

14. Apparatus in accordance with claim 10, wherein the geometrical structure is a plane which is spanned by three prominent points of the body.
